(19) United States
(12) Patent Application Publication
(10) Pub. No.: US 2025/0329419 A1
(43) Pub. Date: Oct. 23, 2025

(54) METHOD AND SYSTEM FOR DETERMINING PEPTIDE FITNESS

(71) Applicants: Gergely KATONA, Göteborg (SE); Maria SJÖGREN BOKAREVA, Göteborg (SE)

(72) Inventors: Gergely KATONA, Göteborg (SE); Maria SJÖGREN BOKAREVA, Göteborg (SE)

(21) Appl. No.: 18/857,759

(22) PCT Filed: Apr. 18, 2023
(86) PCT No.: PCT/SE2023/050351

§ 371 Date: Oct. 17, 2024

(30) Foreign Application Priority Data
Apr. 25, 2022 (SE) 2250492-2

(51) Int. Cl. G16B 40/20; G16B 35/20

(52) U.S. Cl. CPC G16B 40/20; G16B 35/20

(57) ABSTRACT

A method for determining a fitness value of a new peptide including: generating a library of sample peptides having unique amino acid sequences; measuring the interaction of each sample peptide with the target peptide; classifying each of the sample peptides according to their atom type composition, wherein the atom type composition is based on at least one of: the type of element, number of atoms, role in a functional group, position in within the amino acid, or a combination thereof; training a machine learning system with the sample peptides, the training is based on the measured interaction and the atom type composition; providing to the machine learning system a new peptide, not being part of the library of sample peptides; and, predicting via the machine learning system, the fitness value of the new peptide based on the atom type composition of the new peptide.

### FIELD OF THE INVENTION

The present disclosure relates to a method for determining peptide fitness based on atom type composition. In particular it relates to a machine learning system for determining peptide fitness of a new peptide based on atom type composition analysis of a library of physically tested known peptides.

### BACKGROUND OF THE INVENTION

Proteins are biological molecules consisting of at least one chain, or sequence, of amino acids. Proteins differ from one another primarily in their composition of amino acids and secondly in their sequence, the differences of compositions and sequences being called “mutations”.

One of the ultimate goals of protein engineering is the design and construction of peptides, enzymes, proteins, or amino acid sequences with desired properties. The desired properties may collectively called be “fitness”.

Such design typically focuses to generate suitable structures that enables “lock-key” type of fit between (usually binary) cognate interaction partners or allow certain degree of structural adaptation upon complex formation (“induced fit”). Additional and improved methods for determining peptide/protein fitness would be advantageous.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a method for determining a fitness value of a new peptide, the fitness value corresponding to at least interaction strength with a target peptide, wherein the new peptide has not been subject to physical interaction testing with the target peptide. The method including: generating a library of sample peptides having unique amino acid sequences; measuring the interaction of each sample peptide with the target peptide, to determine an interaction value for each of the sample peptides; classifying each of the sample peptides according to their atom type composition, wherein the atom type composition is based on at least one of: the type of element, number of atoms, role in a functional group, position in within the amino acid, or a combination thereof; training a machine learning system with the sample peptides, wherein the training is based on the measured interaction and the atom type composition; providing to the machine learning system a new peptide, not being part of the library of sample peptides; and, predicting via the machine learning system, the fitness value of the new peptide based on the atom type composition of the new peptide.

Also provided is a method for determining atom type composition of a new peptide. The new peptide having a desired fitness, the fitness corresponding to at least interaction strength with a target peptide.

Further advantageous embodiments are disclosed in the appended and dependent patent claims.

### BRIEF DESCRIPTION OF THE DRAWINGS

These and other aspects, features and advantages of which the invention is capable will be apparent and elucidated from the following description of embodiments of the present invention, reference being made to the accompanying drawings, in which

FIG. 1 shows contrasting sequence and atom composition analysis of peptide library derived from proteins in the human proteome. An example peptide PYAPLGTVYRELQKL can be described as a sequence of numbers equivalent to the type of amino acid in the order of the peptide sequence from the N to the C terminus (left), or alternatively the number of amino acids of each type it contains (right). Repeating the feature generation process for a library of peptides and projecting the features with t-distributed stochastic neighbour embedding (T-SNE) results in clear clustering of the features derived from atom composition analysis but unclear clustering based on sequence analysis. True (cyan) and false (red) indicate the ability or lack of ability of survivin to bind to the peptide.

FIG. 2 shows raw fluorescence scans of the peptide microarray (A) incubated with 1 μg/mL survivin and labelled anti-His tag antibody (B) only labelled anti-His tag antibody.

FIG. 3 shows clustering of peptides based on their atom type abundance (light/dark high/low abundance of atom types, respectively, z-score). The grayscale bar indicates the logarithm of fluorescence intensity of the peptide in the survivin peptide microarray experiment (black zero intensity, white highest level of intensity). The prediction bar shows the success of the machine learning prediction using the atom type abundance as features (black and cyan colours mark predicted non-interacting and interacting peptides, respectively).

FIG. 4 shows a detailed look at the relationship between survivin interaction with peptides and peptide composition. A magnified view of the cluster, revealing individual peptides. The heat map depicts the light/dark high/low abundance of atom types (z-score). The grayscale bar represents the logarithm of the peptide's fluorescence intensity in the survivin peptide microarray experiment (black zero intensity. white highest level of intensity). The prediction bar indicates the accuracy of the machine learning prediction using atom type abundance as features (black and cyan colours mark predicted non-interacting and interacting peptides, respectively).

FIG. 5 shows a minimal neural network with nearly identical performance as more complex architectures.

### DETAILED DESCRIPTION

Instead of (primary, secondary, and tertiary) structural description of peptides, this invention focuses on the atom composition of the peptides to make more efficient prediction of peptide fitness.

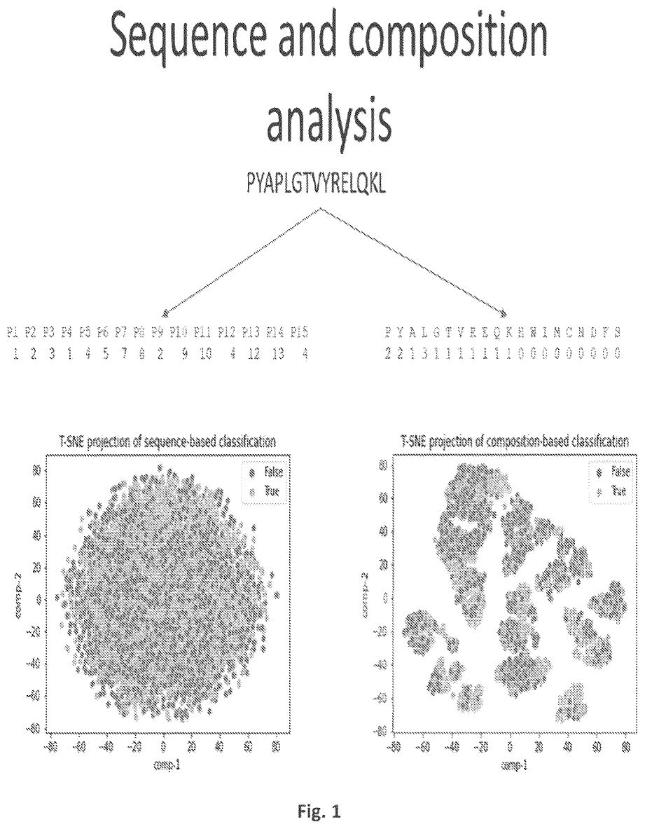

Although all information is already contained in the amino acid sequence, machine learning tools frequently require a large data set and complex modelling layout to approximate even a simple function that internally converts an amino acid sequence to an atom composition without explicit instructions. Classification efficiency can be dramatically improved by using more appropriate features (FIG. 1), which require less training and simpler models. The principle “Occam's razor” also implies that it is preferable to keep the simpler of two models or explanations.

The construction of modified amino acid sequences with engineered amino acid substitutions, deletions or insertions of amino acids or blocks of amino acids (chimeric proteins) (i.e. “mutants”) enables an assessment of the role of any particular atom composition in fitness as well as an understanding of the relationships between the peptide atom composition and its fitness.

The primary goal of quantitative atom composition-function/fitness relationship analysis is to investigate and mathematically describe the effect of peptide composition changes on fitness. The effect of mutations is related to physicochemical and other molecular properties of varying atom composition and can be approached statistically.

Modern machine learning approaches rely heavily on the amount of data available and the best use of difficult-to-obtain data. A peptide microarray experiment, for example, can increase the number of parallel trials, but scaling it to millions of experiments is difficult, and even upscaling does not significantly reduce the sparseness of the data. The number of different 15 amino acid residue long peptide sequences that can be synthesized is enormous: 20¹⁵=3.3×10¹⁹. In a realistic experiment with 10⁴ synthesized peptides, every sample must describe and extrapolate to about 3.3×10¹⁹/10⁴=3.3×10¹⁵ other peptides that were not included in the experiment. In other words, if the sequence space is considered, the modelling will be based on extremely sparse data. Instead of mapping the amino acid composition space, this invention defines a more useful proxy. A 15 amino acid residue long peptide still have (15+20−1)!/15!/(20−1)!=1.86×10⁹ different kind of amino acid composition. Finding similarities in the atom composition of different amino acids can help to narrow down the possible variants even more. The difference in the number of possible composition variants of at least ten orders of magnitude makes sampling substantially less sparse and extrapolation from observed data points much easier. It is certainly possible with current technology to sample at least one unique amino acid composition of 5 amino acid long peptides ((5+20−1)!/5!/(20−1)!=42,504 alternatives). Naturally, which permutation represents a given composition will be arbitrary or based on existing biological sequences. Even if sequence-based modelling is superior, which the present inventors consider there is no evidence of, the computational advantages of atom composition modelling make it appealing to virtually pre-screen peptides and focus on peptide sequences with suitable composition.

The invention can be motivated by the empirical observations in chemistry that led to the establishment of the empirical law “like dissolves like,” and it extends it to fitness predictions in biological interactions. In the field of chemistry, a molecule's ability to dissolve in a specific type of solvent is not primarily determined by its global structure, shape, size, and bonding connectivity. The primary prediction strategies focus on identifying the type and number of so called “functional groups”. A functional group is defined as a collection of identical or different elements that is associated with a localized, relatively rigid electronic structure. For example, to predict the water solubility of organic molecules containing carbon and oxygen atoms, the C/O ratio can be used as a primary predictor [1] although exceptions from this trend exist [2]. Polyethylene glycol (PEG) is miscible with water because both PEG and water contain a large number of oxygen atoms, whereas a hydrocarbon is soluble in other hydrocarbons because both contain a large number of carbon atoms (in typically CH2 groups). When a hydrocarbon is modified by adding one ether group, the modified molecule is not immediately water soluble/miscible. To prevent spontaneous phase separation between an oily (containing the modified hydrocarbon) and a watery phase, more than one ether group is most likely required, as is a C/O atom ratio below a certain threshold (containing mostly water). The position of the added ether groups is not the most important predictor of molecule solubility/partitioning. Furthermore, in complex chemical environments, “likeness” is not a binary choice; many atom types can define a wide range of potential phases for molecules containing these atoms to separate or partition. Polytetrafluorethylene (PTFE, Teflon) coating, for example, contains fluorine atoms and repels both oily and watery substances.

Peptides and proteins typically contain 20 naturally occurring amino acids and a much smaller subset of available functional groups (C, CH, CH2, CH3, hydroxyl, phenyl, carboxyl, amide, sulfhydryl group, and so on) that are present in varying ratios in different amino acids. As a result, the same strategy (counting atoms of a specific type) can be used to predict fitness in a biological context as it can for predicting miscibility/solubility (or, conversely, phase separation) in a chemical context. The question then becomes not whether a peptide is hydrophobic or hydrophilic, but which peptide is dissolved (localized) in the same phase as another. Peptides and proteins act as both solutes and solvents for one another. The question can be rephrased in a biochemical context by asking how atom type composition of peptides determines their spontaneous reactions, localization, and formation of spatially distinct compartments, or other fitness. It is important to note that a peptide with fewer than 20 amino acid residues will lack at least one amino acid type and may lack distinct classes of functional groups.

Unlike docking methods, composition-based modelling does not require a 3D representation of the peptide. Many proteins are intrinsically disordered, limiting the applicability of structure-based modelling, but the present invention does not necessitate knowledge about the primary, secondary, tertiary, and quaternary structures of the partners.

Because even a short peptide has a large sequence space, fitness predictions are usually limited to sequence neighbours. A predicted effect of a point mutation is one example. This invention allows for the generation of accurate predictions about any arbitrary sequence on an absolute scale rather than a relative to a native or wild-type sequence.

### EXPERIMENTAL EXAMPLES

### Peptide Microarray

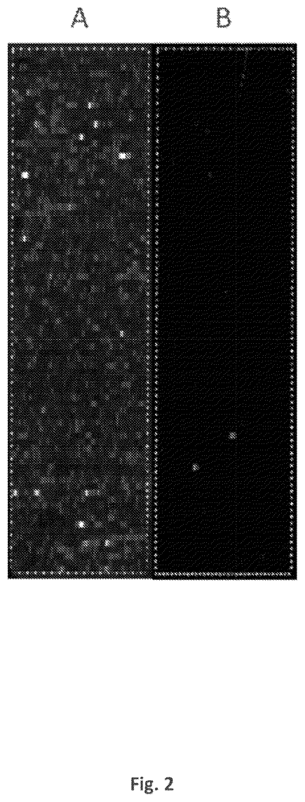

A peptide microarray was designed using the protein sequences from: Cdk1 (P06493), KAT2A/GCN5 (Q92830), SP11/PU1 (P17947), SUZ12 (Q15022), EED (075530), JADE3 (Q92613), DIABLO/SMAC (Q9NR28), BOREALIN (Q53HL2), INCENP (Q9NQS7), SGOL1 (Q5FBB7), SGOL2 (Q562F6), EZH2 (Q15910), JARID2 (Q92833), Histone H3 (P68431), AURORAKB (Q96GD4), JADE1 (Q6|E81), JTB (076095), EVI5 (060447), RAN (P62826), USP9X (Q93008), C-IAP1 (Q13490), STAT3 (P40763), BRUCE/APOLLON (Q9NR09), XPO1 (014980), CDX2 (Q99626), Msx2 (P35548), RBM15 (Q96T37), PHF21A (Q96BD5), PHF8 (Q9UPP1), DIDO (Q9BTC0), JADE2 (Q9NQC1) and HASPIN (Q8TF76). The Uniprot ID is shown in parenthesis. The protein sequences were divided into peptides of 15 amino acids with an overlap of 10 amino acids. Pre-staining of one of the PEPperCHIP Peptide Microarrays was done with the secondary 6×His Tag Antibody DyLight680 antibody at a dilution of 1:1000 and with monoclonal anti-HA (12CA5)-DyLight800 control antibody at a dilution of 1:1000 to investigate background interactions with the protein-derived peptides that could interfere with the main assays. Subsequent incubation of other peptide microarray copies with survivin at a concentration of 1 μg/ml in incubation buffer was followed by staining with the secondary 6×His Tag Antibody DyLight680 (Rockland Immunochemicals) antibody and the monoclonal anti-HA (12CA5)-DyLight800 control antibody (Rockland Immunochemicals) as well as by read-out at scanning intensities of 7/7 (red/green). HA and His tag control peptides were simultaneously stained as internal quality control to confirm the assay quality and to facilitate grid alignment for data quantification. Read-out was performed with a LI-COR Odyssey Imaging System, while quantification of spot intensities and peptide annotation were done with PepSlide Analyzer. Quantification of spot intensities and peptide annotation were based on the 16-bit gray scale tiff files at scanning intensities of 7/7 that exhibit a higher dynamic range than the 24-bit colorized tiff files shown in FIG. 2.

### Classification by Machine Learning

The machine learning process was implemented using the scikit-learn python library. The features of the peptides were the number of atoms that belong to specific atom type categories. Table 1 shows how the atoms in amino acids were assigned for this study. These were summed after translating each amino acid in the peptide to atom types. The 5388 peptides were divided into equally large training and test sets. The training set was classified as interacting (fluorescence intensity greater than zero) or non-interacting (fluorescence intensity equal to zero). The features were standardized before performing the training. Training was performed with the multi-layer perceptron classifier using the default parameters of scikit-learn. The confusion matrix and prediction accuracy were evaluated by the tools provided by the scikit-learn library.

### Prediction of Survivin-Protein Interaction Governed by Amino Acid Composition.

Because of the large number of peptides (n=5395) on the microarray, machine learning approaches were able to characterize the features that promote a peptide to interact with survivin. On the microarray, approximately 40% of the peptides had fluorescence intensities greater than zero, and approximately 20% of the peptides had fluorescence intensities greater than 1000. Seven peptides with a high histidine content were eliminated because they reacted strongly with the anti-His-tag antibody.

In this microarray experiment, the proportions of interacting and non-interacting peptides are thus reasonably balanced. Rather than focusing on the peptide sequence, the peptides were grouped by the abundance of certain atom types in their amino acids to characterize the chemical/positional nature of the atoms (in this example according to Table 1). The number of atoms in each functional groups/moieties are represented.

To illustrate this strategy, here's an everyday example: when describing something, it is often more effective to tell what they comprise or contain rather than what they are like. We can compare it to taking different medications. Different drugs can have different effects, and it's common to take more than one medication at a time. For instance, if you take insulin and a beta blocker, they can have distinct and separate effects such as reducing blood sugar levels and lowering blood pressure.

Consider a scenario where a red tablet contains insulin and a painkiller, and a blue tablet contains a beta blocker and sugar. However, we don't know the exact composition of these treatments just by their appearance. If we take the red pill and blue pill, we may notice that our blood sugar levels vary based on the amount of treatment we apply. The sugar in the blue pill would increase blood sugar levels, while the insulin in the red pill would decrease them. The order in which we administer these treatments, such as first or second thing in the morning, has no effect on their effectiveness, just as the order of amino acids in a sequence is not the most important factor determining their fitness.

To determine the effects of these treatments more accurately, we can test different doses and apply them in different combinations while monitoring their effects. However, this becomes very difficult if we test it with 20 different pills at different doses, but much easier once we know the exact composition of each treatment, even if we don't know all of their effects. It's essential to note that the colour, shape and taste of the pill is not a useful indicator of its composition, even though these may be the most noticeable differences between the treatments.

For amino acid the names glutamine, phenylalanine or alanine are simply distractive, names which do not tell anything about what they are. Nevertheless, chemists have deconstructed organic molecules into functional groups that are shared by naturally occurring amino acids and amino acids can be described as a combination of functional groups, just like a pill can be described as a combination of different drugs. For instance, despite their similar-sounding names, alanine and phenylalanine actually have very little in common, except for their main chain atoms, which are shared by all amino acids except glycine and proline.

Furthermore, there is a common misconception that glutamates and aspartates are similar solely because they can both be negatively charged. However, this notion overlooks the fact that they have CH2 groups, which they share with a variety of other amino acids, including those presumed to be quite distinct, such as proline, arginine, or leucine. Clearly, the analogy between different drugs in a treatment and functional groups ends here because we do not claim that a functional group has a specific biological effect, but instead link the number of functional groups to peptide fitness.

Breaking down an amino acid into individual atoms is not particularly useful, as functional groups consist of a fixed combination of atoms. For example, a carboxyl group always includes one carbonyl carbon and two carboxyl oxygen atoms. Sorting them into separate categories simply creates two groups with perfectly correlated content, which neither helps nor hinders machine learning techniques. In fact, it only serves to make our descriptions needlessly complex.

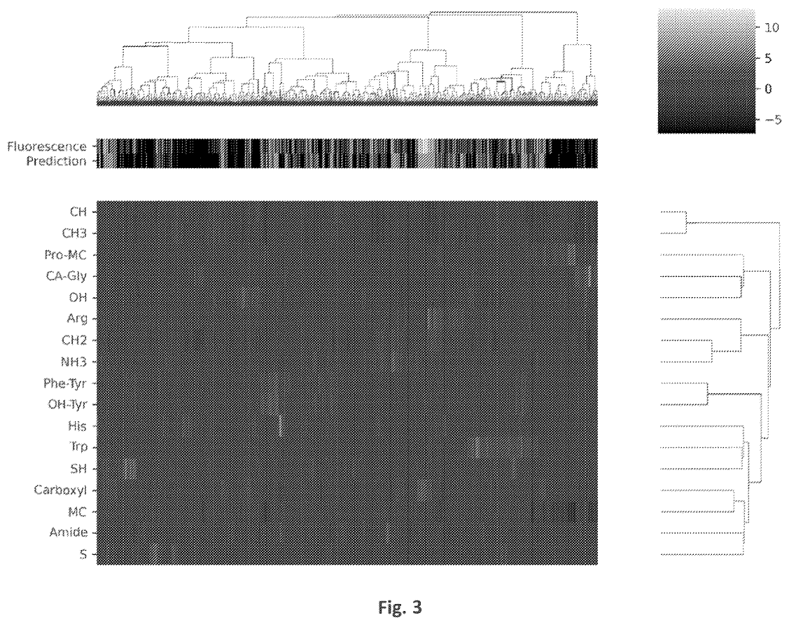

Table 1 shows one method for assigning (non-hydrogen) atoms to “functional group categories” so that their numbers do not correlate with unity. The dendrogram at the top of FIG. 3 can be used to determine how closely they are related. CH and CH3 are the most correlated categories. This is because when a hydrocarbon chain branches, it frequently creates pairs of CH and CH3 groups while removing two CH2 group. Alanine is too short to be branched, so it only contributes a CH3 group without adding a CH, which is one of the reasons why the perfect correlation between CH and CH3 is broken. Methionine also has a terminal CH3 group and is not branched. Despite the fact that Table 1 appears to be a renaming of amino acid names to atom names, the number of categories is only 17, as opposed to the 20 natural amino acid types. Despite the loss of detail, the 17-category version of Table 1 outperforms the alternative in which each atom in an amino acid is assigned to its own category. That is not to say that Table 1 is the only categorisation system, nor necessarily the best, but it serves as a functional example of the present inventive concept.

The inventors have studied the similarity of atomic displacements in protein crystal expecting that it follows the displacement of a classical elastic medium where adjacent atoms share displacement directionality [3, 4]. It was found instead that atoms quite far apart can displace similarly and what these atoms seem to share is their chemical identity. Evidence for collective excitation in protein crystals [5] was identified and the theoretical implications were studied [6].

One such implication of collectiveness is that the number of oscillators has a significant impact on the system's evolution, so counting the number of different oscillators may have a good predictive value. The position of the atoms in the structure, on the other hand, does not appear to matter, so the structure can be ignored as a first approximation. The dynamics of components, which change qualitatively when the system transitions from one phase to another, are also fundamentally dependent on phase transitions. The cooling of water is a useful example. At a sharp transition temperature, liquid water molecules begin to separate to solid regions, where their degrees of freedom are drastically reduced, and their dynamics become lattice fluctuations rather than symmetric free diffusion in a liquid phase. When a fatty acid transitions from a watery to an oily phase, the molecular dynamics undergo a similar but less dramatic change. So far, applying these theoretical biophysical considerations to biochemical practice has shown to function, but the predictive power of this method and its link to collective excitations may be entirely coincidental.

### Detailed Explanation of Table 1

Alanine (A) is described with four atoms in the main chain (MC): carbonyl carbon, carbonyl oxygen, amide nitrogen and alpha carbon (CH). CH3 group as side chain.

Cysteine (C) has the equivalent four atoms in the main chain. The beta carbon is a CH2 atom, and it also has a unique SH group in the reduced form. No alternative is assumed for the different oxidized forms of the sulfur.

Aspartate (D) has the equivalent four atoms in the main chain. The beta carbon is a CH2 atom and it has a carboxyl group (labelled “Carboxyl”) consists of a carbonyl carbon and two oxygen atoms. The protonated form of the side chain is not explicitly assumed. If the two forms of side chain affect the fitness the large number of D and E without positively charged side chains as neighbours may implicitly be encoded as a different kind of side chain by the neural network. This is because large number of negative charges in the vicinity may increase the pKa of the side chain so that a larger fraction of side chains may be in their protonated form instead.

Glutamate (E) Deconstructed similarly as D, with an extra CH2 group in the side chain corresponding to the gamma carbon group.

Phenylalanine (F) has the equivalent four atoms in the main chain, the beta carbon is a CH2 atom and the phenyl group consisting of six aromatic CH groups. The aromatic ring is assumed to have similar properties as the ring of tyrosine (label “Phe-Tyr”).

Glycine (G) has only three equivalent atoms in the main chain: carbonyl carbon, carbonyl oxygen, amide nitrogen. The alpha carbon is a CH2 atom in glycin and it has its separate category as it belongs to the main chain, rather than the side chain.

Histidine (H) has four standard main chain atoms and a CH2 beta carbon. Its indole ring contains five unique non-hydrogen atoms (label “His” in Table 1). The protonation state of the side chain is ignored on the feature level, but clearly the number of E, D, K and R amino acids will have a profound effect on the pKa of histidine and can be implicitly inferred by machine learning training (if the protonation state of histidine affects the fitness).

Isoleucine (I) has four standard main chain atoms and a branched side chain consisting of two CH3, one CH and one CH3 carbons.

Lysine (K) has four standard main chain atoms and a side chain consisting of four CH2 atoms and one amino group (label “NH3”) which is often protonated.

Leucine (L) has four standard main chain atoms and a branched side chain consisting of two CH3, one CH and one CH3 carbons. Table 1 conversion table does not distinguish between leucine and isoleucine.

Methionine (M) has four standard main chain atoms, two CH2 groups a sulfur atom (label “S”) and CH3 terminal carbon forming a thioether group.

Asparagine (N) has four standard main chain atoms, a CH2 beta carbon and an amide group in its side chain consisting of a carbonyl carbon, carbonyl oxygen and a NH2 group (=three non-hydrogen atoms in this functional group with label “Amide”).

Proline (P) is a circular amino acid with special main chain. Only two main chain is considered standard: its carbonyl carbon and carbonyl oxygen in the MC category. The alpha C atom is still a CH atom, but it is much more constrained than in other amino acids. The nitrogen atom is bonded to the side chain, and it is not an NH group like in other amino acids. Therefore, these two atoms are assigned to a special main chain category specific to proline (Pro-MC). The although the side chain is special with its circular connectivity the participating CH2 groups (three of them) are pooled together with other CH2 groups in Table 1.

Glutamine (Q) is related to asparagine, with longer side chain due to an additional CH2 group.

Arginine (R) has a long side chain with 3 CH2 groups and a unique guanidine group with 3 nitrogen and one carbon. These four atoms are marked with label “Arg” in Table 1.

Serine (S) has four standard main chain atoms and a CH2 group for beta carbon and a hydroxyl group (labelled “OH”).

Threonine (T) has four standard main chain atoms. Its beta carbon is a CH group instead and connected to a hydroxyl (“OH”) and a CH3 group.

Tyrosine (Y) has four standard main chain atoms and an aromatic side chain consisting of six carbon atoms (category “Phe-Tyr”) and a hydroxyl group with its own category (label “OH-Tyr”).

Valine (V) has four standard main chain atoms and a branched hydrocarbon side chain consisting of one CH and two CH3 groups.

Tryptophan (W) has four standard main chain atoms and a CH2 beta carbon. It has additional 9 non-hydrogen atoms in a unique, large heterocyclic indole ring, which is labelled “Trp” in Table 1.

| aa | CA-Gly | Pro-MC | Carboxyl | Amide | His | Trp | Phe-Tyr | OH-Tyr | CH2 | CH | CH3 | OH | SH | S | NH3 | Arg | MC |
|---|---|---|---|---|---|---|---|---|---|---|---|---|---|---|---|---|---|
| A | 0 | 0 | 0 | 0 | 0 | 0 | 0 | 0 | 0 | 0 | 1 | 0 | 0 | 0 | 0 | 0 | 4 |
| C | 0 | 0 | 0 | 0 | 0 | 0 | 0 | 0 | 1 | 0 | 0 | 0 | 1 | 0 | 0 | 0 | 4 |
| D | 0 | 0 | 3 | 0 | 0 | 0 | 0 | 0 | 1 | 0 | 0 | 0 | 0 | 0 | 0 | 0 | 4 |
| E | 0 | 0 | 3 | 0 | 0 | 0 | 0 | 0 | 2 | 0 | 0 | 0 | 0 | 0 | 0 | 0 | 4 |
| F | 0 | 0 | 0 | 0 | 0 | 0 | 6 | 0 | 1 | 0 | 0 | 0 | 0 | 0 | 0 | 0 | 4 |
| G | 1 | 0 | 0 | 0 | 0 | 0 | 0 | 0 | 0 | 0 | 0 | 0 | 0 | 0 | 0 | 0 | 3 |
| H | 0 | 0 | 0 | 0 | 5 | 0 | 0 | 0 | 1 | 0 | 0 | 0 | 0 | 0 | 0 | 0 | 4 |
| I | 0 | 0 | 0 | 0 | 0 | 0 | 0 | 0 | 1 | 1 | 2 | 0 | 0 | 0 | 0 | 0 | 4 |
| K | 0 | 0 | 0 | 0 | 0 | 0 | 0 | 0 | 4 | 0 | 0 | 0 | 0 | 0 | 1 | 0 | 4 |
| L | 0 | 0 | 0 | 0 | 0 | 0 | 0 | 0 | 1 | 1 | 2 | 0 | 0 | 0 | 0 | 0 | 4 |
| M | 0 | 0 | 0 | 0 | 0 | 0 | 0 | 0 | 2 | 0 | 1 | 0 | 0 | 1 | 0 | 0 | 4 |
| N | 0 | 0 | 0 | 3 | 0 | 0 | 0 | 0 | 1 | 0 | 0 | 0 | 0 | 0 | 0 | 0 | 4 |
| P | 0 | 2 | 0 | 0 | 0 | 0 | 0 | 0 | 3 | 0 | 0 | 0 | 0 | 0 | 0 | 0 | 2 |
| Q | 0 | 0 | 0 | 3 | 0 | 0 | 0 | 0 | 2 | 0 | 0 | 0 | 0 | 0 | 0 | 0 | 4 |
| R | 0 | 0 | 0 | 0 | 0 | 0 | 0 | 0 | 3 | 0 | 0 | 0 | 0 | 0 | 0 | 4 | 4 |
| S | 0 | 0 | 0 | 0 | 0 | 0 | 0 | 0 | 1 | 0 | 0 | 1 | 0 | 0 | 0 | 0 | 4 |
| T | 0 | 0 | 0 | 0 | 0 | 0 | 0 | 0 | 0 | 1 | 1 | 1 | 0 | 0 | 0 | 0 | 4 |
| Y | 0 | 0 | 0 | 0 | 0 | 0 | 6 | 1 | 1 | 0 | 0 | 0 | 0 | 0 | 0 | 0 | 4 |
| V | 0 | 0 | 0 | 0 | 0 | 0 | 0 | 0 | 0 | 1 | 2 | 0 | 0 | 0 | 0 | 0 | 4 |
| W | 0 | 0 | 0 | 0 | 0 | 9 | 0 | 0 | 1 | 0 | 0 | 0 | 0 | 0 | 0 | 0 | 4 |

### Clustering of Peptides

Firstly, the peptides were clustered using Ward's method based on their atom type composition and abundance. (FIG. 3).

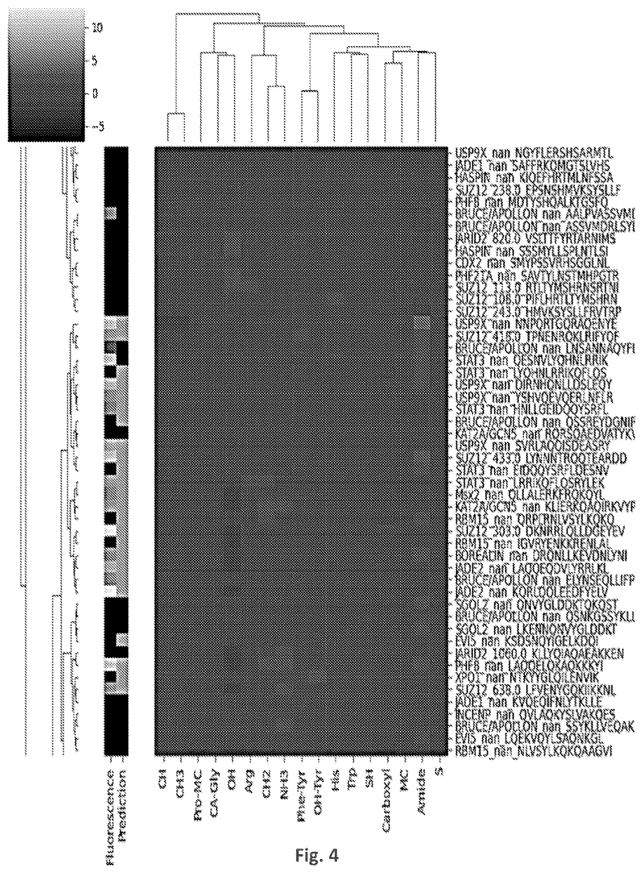

The clustering revealed that the similarity of the peptides in atom type abundance is correlated with the fluorescence intensity caused by survivin interaction. On a larger scale, the accumulation of interacting peptides can be seen, which is linked to specific atom compositions. The dendrogram contains multiple major branches with high frequency of interacting peptides and similarly large branches that are not or are very sparsely populated with interacting peptides. This does not appear to be a simple function of chemical group presence or absence, such as the presence of a large number of carboxyl groups. Simple decision trees, a focus on extremely large net charges, or similar extremes of hydrophobicity are unsuitable for predicting whether a peptide is interacting accurately and sensitively. For a peptide to interact with survivin, a specific combination of atom types must be enhanced/depleted. A magnified section of the cluster (FIG. 4) shows that, despite the different density of interacting peptides in the global dendrogram, the suitability of a peptide for interaction is extremely fine-grained. When interacting with survivin, even peptides with very similar compositions can behave differently.

As a result, clustering based solely on similarity metrics may be insufficient.

To continue the analysis, the peptides were classified as interacting or non-interacting based on the intensity of the associated fluorescence, and a multilayer perceptron classifier was trained on half of the data set to recognize these two classes based on atom composition features. On the remaining half of the data set, the prediction strength was tested. Table 2 depicts the confusion matrix.

TABLE 2
Confusion matrices of predictions.

| | | Predicted | All | | Predicted | Non- |
|---|---|---|---|---|---|---|
| | | label | peptides | | label | overlapping |
| | | Non-interacting | Interacting | | Non-binder | Interacting |
| Correct | Non-interacting | 1422 | 276 | Non-interacting | 464 | 101 |
| label | Interacting | 348 | 652 | Interacting | 119 | 215 |
| Precision/ | | 0.80/0.84 | 0.70/0.65 | | 0.80/0.82 | 0.68/0.64 |
| recall | | | | | | |

In addition, the predicted interacting peptides are highlighted in cyan in FIGS. 2 and 3. Although approximately two-thirds of the interacting peptides (precision 0.70, recall 0.65) were correctly recognized, the prediction specificity and sensitivity appeared to be the most successful for the non-interacting peptides (precision 0.80, recall 0.84), providing a solid foundation for a negative selection of the peptides. The predictions follow the fluorescence signal in FIG. 2 remarkably well, and the network frequently correctly predicts the interacting peptides even among very similar peptides on a more local scale (FIG. 3). Because the peptides overlap in sequence, the test set could be contaminated by the sequence similarity of the training set. We eliminated the effect of overlaps by considering only every third peptide in the test and training sets. The relative proportions of elements in the confusion matrix are very similar to the relative proportions of elements in the confusion matrix when every peptide was classified. As a result, we concluded that the prediction strength did not result from the training set contaminating the test set. When interacting peptides with fluorescence intensities less than 100 were removed from the training and testing sets, the prediction of interacting peptides did not improve. (Table 3)

TABLE 3
Confusion matrices of predictions with weak interaction partners (0 <
Fluorescence intensity (FI) < 100) excluded from the analysis.

| | | Predicted | All peptides | | Predicted | Non-overlapping |
|---|---|---|---|---|---|---|
| | | label | FI >100 or =0 | | label | FI >100 or =0 |
| | | Non-interacting | Interacting | | Non-interacting | Interacting |
| Correct | Non-interacting | 1450 | 282 | Non-interacting | 477 | 93 |
| label | Interacting | 297 | 610 | Interacting | 105 | 205 |
| Precision/ | | 0.83/0.84 | 0.68/0.67 | | 0.82/0.84 | 0.69/0.66 |
| recall | | | | | | |

The distinctness of peptide features as described by amino acid composition was assessed. Only eight peptides were identified with at least one pair with identical amino acid composition, which, once again, cannot explain the robustness of predictions. The near exclusive uniqueness of peptides is expected from a random model, which assumes uniform probability of the 20 amino acids to occur at any position in the 15 amino acid long peptide. The probability of obtaining a specific peptide sequence is

$$\frac{1}{2 {0}^{15}} = 3.1 \times 1 {0}^{- 20} ,$$

but the abundance of amino acids is not specific to a particular sequence. The average probability of obtaining a signature composition in a 15 amino acid peptide can be approximated using the inverse combination with repetition

$$( \frac{15 ! ( 20 - 1 ) !}{( 15 + 20 - 1 ) !} = 5.4 \times 1 {0}^{- 10} ) .$$

Clearly, this will be an extremely asymmetric distribution, with certain combinations, such as 15 identical amino acids, being orders of magnitude less frequent than others. A multinomial distribution with parameters 15 trials with uniform probability of 0.05 for each of the 20 amino acids can describe the entire multivariate distribution. This distinction between sequence and composition necessitates a reconsideration of uniqueness of a biological sequence. While the appearance of an arbitrary peptide sequence has the same a priori probability, the appearance of a peptide with biased composition is a priori unlikely and should be unexpected in biological context. This is in stark contrast to how repeating/biased composition sequences are referred to as “redundant”, “low complexity” or “low information content”. Perhaps this misconception stems from IT-technology, where identical bytes can be compressed to increase data storage efficiency, but it is completely irrelevant in biological context. This inventive concept described herein intends to consider the very uneven distribution of composition space by locating, highlighting, and oversampling the rare sequences with high composition bias while drastically under sampling the common permutations of truly redundant sequences for screening purposes.

In this microarray design, 10 amino acids were constrained between adjacent peptides, reducing peptide variability even further. Adjacent peptides with identical amino acid content are still very rare using the random model

$$( \frac{5 ! ( 20 - 1 ) !}{( 5 + 20 - 1 ) !} = 2.4 \times 1 {0}^{- 5}$$

in an array of 5395 peptides). Nonetheless, functional protein sequences are not evolved at random, as evidenced by the frequent occurrence of sequences with a high compositional bias.

Because relatively few peptides in the microarray have completely independent binding strength from their neighbours, one can argue that the minimum length of a peptide required to elicit survivin binding is equal to or less than 5 amino acids. This length is consistent with the length of “signal peptides” such as the nuclear localization signal, which also exhibits strong compositional bias, such as peptide PKKKRKV in the SV40 Large T-antigen. Non-randomness also explains the presence of a minor fraction of peptides with identical amino acid compositions, and such repeated occurrences were only associated with polypeptide chains from the same protein.

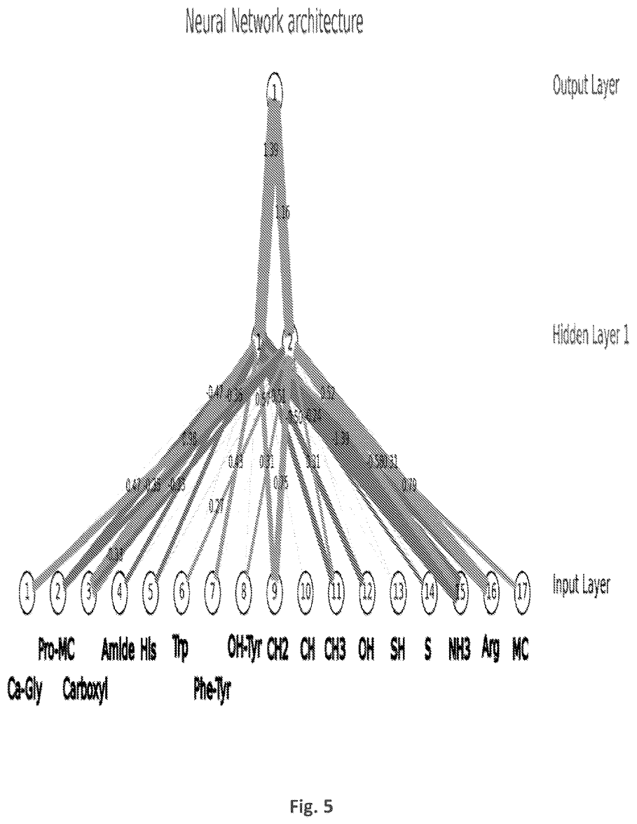

It is also possible to dramatically reduce the network's complexity to just two hidden layer perceptrons and visualize the weight of all network edges. (FIG. 5) It is worth noting that the 17 atom categories are fewer than the 20 conventional amino acids, which reduces feature dimensionality without sacrificing accuracy. The colours orange and blue between the input and hidden layers indicate a positive and negative contribution to survivin binding, respectively.

The simplicity of the network makes the binding phenomenon understandable to humans. For example: “Carboxyl and amino groups are favourable for survivin binding, but not when they are present at the same time”, “Hydroxyl groups are tolerated together with lysine residues, but not favourable together with carboxyl groups.”, “Trp residues work well together with Arg or Lys residues, but are indifferent when carboxyl groups are present.”, “If there is a choice between a shorter and longer amino acid such as aspartate or glutamate, the longer one will be more favourable for binding because it contains more CH2 groups.” The prediction of this simple network is still a fine-grained decision based on the exact atom/amino acid content.

### Prediction of Protein Binding to Survivin Fitness in the Proteome

A trained neural network can predict survivin binding to any 15 amino acid long peptide, including all human proteome sequence fragments. Table 4 displays the top 80 predicted survivin binders, excluding short proteins. Notably, nuclear, chromatin-associated proteins, and mitochondrial proteins, including members of the respiratory chain, are abundant. It is well established that survivin preferentially localizes in nuclear and mitochondrial compartments and that survivin influences oxidative phosphorylation. [7, 8]

TABLE 4
Predicted strongly binding proteins to survivin in the human proteome.

| | | Fraction of | |
|---|---|---|---|
| | | PF that | |
| | Peptide | predicted to | Uniprot |
| Protein names | fragments (PF) | bind | Entry |
| 26S proteasome complex subunit SEM1 | 11 | 1.00 | P60896 |
| (26S proteasome complex subunit DSS1) | | | |
| (Deleted in split hand/split foot protein 1) | | | |
| (Split hand/foot deleted protein 1) (Split | | | |
| hand/foot malformation type 1 protein) | | | |
| Keratin-associated protein 22-2 | 6 | 1.00 | Q9BYT5 |
| Minor histocompatibility protein HB-1 | 6 | 1.00 | Q4G0Z9 |
| [Cleaved into: Minor histocompatibility | | | |
| antigen HB-1 (mHag HB-1)] | | | |
| Protein FAM240A | 14 | 1.00 | A0A1B0GTK4 |
| Sperm protamine P1 (Cysteine-rich | 8 | 1.00 | Q9C093 |
| protamine) | | | |
| Non-histone chromosomal protein HMG-17 | 15 | 0.93 | Q5T1S8 |
| (High mobility group nucleosome-binding | | | |
| domain-containing protein 2) | | | |
| Small integral membrane protein 31 | 12 | 0.92 | Q9BZL3 |
| MIEF1 upstream open reading frame protein | 11 | 0.91 | P21741 |
| (Alternative MIEF1 protein) (AltMIEF1) | | | |
| (MIEF1 microprotein) (MIEF1-MP) | | | |
| Keratin-associated protein 19-8 | 10 | 0.90 | Q3LI70 |
| Prothymosin alpha [Cleaved into: | 20 | 0.90 | Q04941 |
| Prothymosin alpha, N-terminally processed; | | | |
| Thymosin alpha-1] | | | |
| Trichohyalin | 386 | 0.90 | Q5T2D2 |
| Embryonic testis differentiation protein | 9 | 0.89 | Q3ZM63 |
| homolog A | | | |
| Embryonic testis differentiation protein | 9 | 0.89 | P0DPP9 |
| homolog B | | | |
| Parathymosin | 18 | 0.89 | Q9UQ90 |
| Protamine-2 (Sperm histone P2) (Sperm | 18 | 0.89 | P15309 |
| protamine P2) [Cleaved into: Basic nuclear | | | |
| protein HPI1; Basic nuclear protein HPI2; | | | |
| Basic nuclear protein HPS1; Basic nuclear | | | |
| protein HPS2; Sperm histone HP4 (Sperm | | | |
| protamine P4); Sperm histone HP2 (Sperm | | | |
| protamine P2) (P2′); Sperm histone HP3 | | | |
| (P2″) (Sperm protamine P3)] | | | |
| Non-histone chromosomal protein HMG-14 | 17 | 0.88 | Q13253 |
| (High mobility group nucleosome-binding | | | |
| domain-containing protein 1) | | | |
| Cerebellar degeneration-related antigen 1 | 50 | 0.88 | P51861 |
| (CDR34) | | | |
| ATP synthase subunit epsilon-like protein, | 8 | 0.88 | Q5VTU8 |
| mitochondrial (ATP synthase F1 subunit | | | |
| epsilon pseudogene 2) | | | |
| Complexin-1 (Complexin I) (CPX I) | 24 | 0.88 | O14810 |
| (Synaphin-2) | | | |
| Spermatid nuclear transition protein 1 (STP- | 8 | 0.88 | Q99932 |
| 1) (TP-1) | | | |
| Coiled-coil domain-containing protein 12 | 31 | 0.87 | Q8WUD4 |
| High mobility group nucleosome-binding | 54 | 0.87 | P82970 |
| domain-containing protein 5 (Nucleosome- | | | |
| binding protein 1) | | | |
| Electron transfer flavoprotein regulatory | 15 | 0.87 | Q6IPR1 |
| factor 1 (LYR motif-containing protein 5) | | | |
| Meiosis expressed gene 1 protein homolog | 15 | 0.87 | P42679 |
| Serine/arginine-rich splicing factor 4 (Pre- | 96 | 0.86 | Q01130 |
| mRNA-splicing factor SRP75) (SRP001LB) | | | |
| (Splicing factor, arginine/serine-rich 4) | | | |
| Troponin C, skeletal muscle | 29 | 0.86 | P28289 |
| RNA guanine-N7 methyltransferase | 21 | 0.86 | Q9GZR2 |
| activating subunit (Protein FAM103A1) (RNA | | | |
| guanine-7 methyltransferase activating | | | |
| subunit) (RNMT-activating mRNA cap | | | |
| mini protein) (RAM) | | | |
| methyltransferase | | | |
| subunit) (RNMT-activating | | | |
| mini protein) (RAM) | | | |
| U4/U6.U5 small nuclear ribonucleoprotein | 28 | 0.86 | Q9Y5J1 |
| 27 kDa protein (U4/U6.U5 snRNP 27 kDa | | | |
| protein) (U4/U6.U5-27K) (Nucleic acid- | | | |
| binding protein RY-1) (U4/U6.U5 tri-snRNP- | | | |
| associated 27 kDa protein) (27K) (U4/U6.U5 | | | |
| tri-snRNP-associated protein 3) | | | |
| Calumenin (Crocalbin) (IEF SSP 9302) | 60 | 0.85 | O43852 |
| Cytochrome b-c1 complex subunit 7 | 20 | 0.85 | P14927 |
| (Complex III subunit 7) (Complex III subunit | | | |
| VII) (QP-C) (Ubiquinol-cytochrome c | | | |
| reductase complex 14 kDa protein) | | | |
| Small integral membrane protein 40 | 13 | 0.85 | A0A1B0GW54 |
| MORN repeat-containing protein 3 | 45 | 0.84 | Q5T089 |
| Serine/arginine-rich splicing factor 10 (40 | 50 | 0.84 | Q96IZ7 |
| kDa SR-repressor protein) (SRrp40) (FUS- | | | |
| interacting serine-arginine-rich protein 1) | | | |
| (Splicing factor SRp38) (Splicing factor, | | | |
| arginine/serine-rich 13A) (TLS-associated | | | |
| protein with Ser-Arg repeats) (TASR) (TLS- | | | |
| associated protein with SR repeats) (TLS- | | | |
| associated serine-arginine protein) (TLS- | | | |
| associated SR protein) | | | |
| Cylicin-2 (Cylicin II) (Multiple-band | 67 | 0.84 | Q14093 |
| polypeptide II) | | | |
| Anaphase-promoting complex subunit 13 | 12 | 0.83 | Q9BS18 |
| (APC13) (Cyclosome subunit 13) | | | |
| Beta-defensin 104 (Beta-defensin 4) (BD-4) | 12 | 0.83 | Q8WTQ1 |
| (DEFB-4) (hBD-4) (Defensin, beta 104) | | | |
| Guanine nucleotide-binding protein G(T) | 12 | 0.83 | P63211 |
| subunit gamma-T1 (Transducin gamma | | | |
| chain) | | | |
| Keratin-associated protein 20-3 | 6 | 0.83 | Q3LI63 |
| Maturin (Maturin neural progenitor | 24 | 0.83 | Q9NR99 |
| differentiation regulator protein homolog) | | | |
| (Protein Ells1) | | | |
| Proline-rich protein 15-like protein (Protein | 18 | 0.83 | Q9BWN1 |
| ATAD4) | | | |
| Protein PET100 homolog, mitochondrial | 12 | 0.83 | Q9BRX2 |
| Putative 60S ribosomal protein L13a protein | 18 | 0.83 | Q9NQ39 |
| RPL13AP3 (60S ribosomal protein L13a | | | |
| pseudogene 3) | | | |
| Serine/arginine-rich splicing factor 6 (Pre- | 66 | 0.83 | Q08170 |
| mRNA-splicing factor SRP55) (Splicing | | | |
| factor, arginine/serine-rich 6) | | | |
| Thymosin beta-10 | 6 | 0.83 | Q969D9 |
| Nuclear cap-binding protein subunit 2 (20 | 29 | 0.83 | Q9H930 |
| kDa nuclear cap-binding protein) (Cell | | | |
| proliferation-inducing gene 55 protein) | | | |
| (NCBP 20 kDa subunit) (CBP20) (NCBP- | | | |
| interacting protein 1) (NIP1) | | | |
| EKC/KEOPS complex subunit GON7 | 17 | 0.82 | Q9BXV9 |
| Transcription elongation factor A protein-like | 17 | 0.82 | Q15560 |
| 7 (TCEA-like protein 7) (Transcription | | | |
| elongation factor S-II protein-like 7) | | | |
| 60S ribosomal protein L38 (Large ribosomal | 11 | 0.82 | P63173 |
| subunit protein eL38) | | | |
| Guanine nucleotide-binding protein | 11 | 0.82 | O14610 |
| G(I)/G(S)/G(O) subunit gamma-T2 (G | | | |
| gamma-C) (G-gamma-8) (G-gamma-9) | | | |
| (Guanine nucleotide binding protein gamma | | | |
| transducing activity polypeptide 2) | | | |
| Putative uncharacterized protein PRO0255 | 11 | 0.82 | Q6XCG6 |
| Splicing factor 3B subunit 6 (Pre-mRNA | 22 | 0.82 | Q15427 |
| branch site protein p14) (SF3b 14 kDa | | | |
| subunit) (SF3B14a) (Spliceosome- | | | |
| associated protein, 14-kDa) (Splicing factor | | | |
| 3b, subunit 6, 14kDa) | | | |
| Transcription initiation factor TFIID subunit | 22 | 0.82 | P52655 |
| 13 (Transcription initiation factor TFIID 18 | | | |
| kDa subunit) (TAF(II)18) (TAFII-18) | | | |
| (TAFII18) | | | |
| Sarcoplasmic reticulum histidine-rich | 137 | 0.82 | O00631 |
| calcium-binding protein | | | |
| Protein FRA10AC1 | 60 | 0.82 | Q96HJ9 |
| Pleckstrin homology domain-containing | 27 | 0.81 | Q8IVE3 |
| family J member 1 (PH domain-containing | | | |
| family J member 1) (Guanine nucleotide- | | | |
| releasing protein x) | | | |
| Cold-inducible RNA-binding protein (A18 | 32 | 0.81 | Q14011 |
| hnRNP) (Glycine-rich RNA-binding protein | | | |
| CIRP) | | | |
| Cytochrome b-c1 complex subunit 6, | 16 | 0.81 | P07919 |
| mitochondrial (Complex III subunit 6) | | | |
| (Complex III subunit VIII) (Cytochrome c1 | | | |
| non-heme 11 kDa protein) (Mitochondrial | | | |
| hinge protein) (Ubiquinol-cytochrome c | | | |
| reductase complex 11 kDa protein) | | | |
| Gamma-crystallin C (Gamma-C-crystallin) | 32 | 0.81 | P07315 |
| (Gamma-crystallin 2-1) (Gamma-crystallin 3) | | | |
| Protein canopy homolog 1 | 16 | 0.81 | Q9BWL3 |
| DNA-directed RNA polymerase III subunit | 42 | 0.81 | O15318 |
| RPC7 (RNA polymerase III subunit C7) | | | |
| (DNA-directed RNA polymerase III subunit | | | |
| G) (RNA polymerase III 32 kDa apha | | | |
| subunit) (RPC32-alpha) (RNA polymerase III | | | |
| 32 kDa subunit) (RPC32) | | | |
| Protein FAM133A | 47 | 0.81 | Q86XD5 |
| Arginine and glutamate-rich protein 1 | 52 | 0.81 | Q9NWB6 |
| Serine/arginine-rich splicing factor 5 | 52 | 0.81 | P84103 |
| (Delayed-early protein HRS) (Pre-mRNA- | | | |
| splicing factor SRP40) (Splicing factor, | | | |
| arginine/serine-rich 5) | | | |
| Centrin-3 | 31 | 0.81 | 015182 |
| Nuclear ubiquitous casein and cyclin- | 46 | 0.80 | Q05952 |
| dependent kinase substrate 1 (P1) | | | |
| Troponin T, fast skeletal muscle (TnTf) | 51 | 0.80 | P67936 |
| (Beta-TnTF) (Fast skeletal muscle troponin | | | |
| T) (fTnT) | | | |
| Calmodulin regulator protein PCP4 (Brain- | 10 | 0.80 | P48539 |
| specific polypeptide PEP-19) (Purkinje cell | | | |
| protein 4) | | | |
| High mobility group nucleosome-binding | 15 | 0.80 | O00479 |
| domain-containing protein 4 (Non-histone | | | |
| chromosomal protein HMG-17-like 3) (Non- | | | |
| histone chromosomal protein) | | | |
| Transcription elongation factor A protein-like | 40 | 0.80 | Q8N8B7 |
| 4 (TCEA-like protein 4) (Transcription | | | |
| elongation factor S-II protein-like 4) | | | |
| Tropomyosin alpha-1 chain (Alpha- | 54 | 0.80 | Q8NBA8 |
| tropomyosin) (Tropomyosin-1) | | | |
| Tropomyosin beta chain (Beta-tropomyosin) | 54 | 0.80 | PODKB5 |
| (Tropomyosin-2) | | | |
| Eukaryotic translation initiation factor 3 | 49 | 0.80 | O75822 |
| subunit J (elF3j) (Eukaryotic translation | | | |
| initiation factor 3 subunit 1) (elF-3-alpha) | | | |
| (elF3 p35) | | | |

Prediction of which Mutations in Peptides Change Binding to Survivin

Table 5 depicts the predicted effect of mutations in two peptides. Replacing a L with a carboxyl-containing amino acid (E or D), arginine (but not K), or a specific type of aromatic amino acid (W, Y, but not F or H) will most likely convert the first peptide to a binder. The second peptide is predicted to be a binder in its wild-type form, but removing a favorable glutamate does not necessarily prevent binding. Glutamine mutant is still a likely binder, but not asparagine, because it has one fewer CH2 group. It should be noted that the input does not have a separate concept for glutamine and asparagine; these terms are only understood as having slightly different atom compositions. Every altered peptide is converted to an atom composition description.

TABLE 5
Mutational analysis of two peptides
(one predicted to be binder and one non-binder).

| Peptide | Position | Mutation | Binder |
|---|---|---|---|
| EYAPLGTVYRELQKP | 225 | L225P | FALSE |
| EYAPLGTVYRELQKT | 225 | L225T | FALSE |
| EYAPLGTVYRELQKS | 225 | L225S | FALSE |
| EYAPLGTVYRELQKY | 225 | L225Y | TRUE |
| EYAPLGTVYRELQKW | 225 | L225W | TRUE |
| EYAPLGTVYRELQKF | 225 | L225F | FALSE |
| EYAPLGTVYRELQKE | 225 | L225E | TRUE |
| EYAPLGTVYRELQKD | 225 | L225D | TRUE |
| EYAPLGTVYRELQKG | 225 | L225G | FALSE |
| EYAPLGTVYRELQKM | 225 | L225M | FALSE |
| EYAPLGTVYRELQKQ | 225 | L225Q | FALSE |
| EYAPLGTVYRELQKH | 225 | L225H | FALSE |
| EYAPLGTVYRELQKA | 225 | L225A | FALSE |
| EYAPLGTVYRELQKR | 225 | L225R | TRUE |
| EYAPLGTVYRELQKV | 225 | L225V | FALSE |
| EYAPLGTVYRELQKN | 225 | L225N | FALSE |
| EYAPLGTVYRELQKK | 225 | L225K | FALSE |
| EYAPLGTVYRELQKC | 225 | L225C | FALSE |
| EYAPLGTVYRELQKI | 225 | L225I | FALSE |
| EYAPLGTVYRELQKL | 0 | WT | FALSE |
| GTVYRELQKLSKFDP | 230 | E230P | FALSE |
| GTVYRELQKLSKFDL | 230 | E230L | FALSE |
| GTVYRELQKLSKFDT | 230 | E230T | FALSE |
| GTVYRELQKLSKFDS | 230 | E230S | FALSE |
| GTVYRELQKLSKFDY | 230 | E230Y | TRUE |
| GTVYRELQKLSKFDW | 230 | E230W | TRUE |
| GTVYRELQKLSKFDF | 230 | E230F | TRUE |
| GTVYRELQKLSKFDD | 230 | E230D | TRUE |
| GTVYRELQKLSKFDG | 230 | E230G | TRUE |
| GTVYRELQKLSKFDM | 230 | E230M | FALSE |
| GTVYRELQKLSKFDQ | 230 | E230Q | TRUE |
| GTVYRELQKLSKFDH | 230 | E230H | FALSE |
| GTVYRELQKLSKFDA | 230 | E230A | FALSE |
| GTVYRELQKLSKFDR | 230 | E230R | TRUE |
| GTVYRELQKLSKFDV | 230 | E230V | FALSE |
| GTVYRELQKLSKFDN | 230 | E230N | FALSE |
| GTVYRELQKLSKFDK | 230 | E230K | TRUE |
| GTVYRELQKLSKFDC | 230 | E230C | TRUE |
| GTVYRELQKLSKFDI | 230 | E230I | FALSE |
| GTVYRELQKLSKFDE | 0 | WT | TRUE |

A similar mutational analysis can be performed on longer sequences to determine which type of mutation is most likely to cause binding in this range (or non-binding). For example, a 30 amino acid region was converted into four 15 amino acid long peptides containing 10 amino acid overlaps. Every amino acid position was changed, and the peptide was tested to see if it was predicted to be a binder or a non-binder. Mutations that change E to M, A, T, S, H, V, P, I, L, or N are the most likely to eliminate binding. To prevent binding, L can also be mutated to P. (Table 6)

TABLE 6
The type of mutations that most likely convert a peptide to a survivin
binder or non-binder. Only a subset of mutation types is displayed.

| Mutation | | |
|---|---|---|
| type | non-binder | binder |
| EM | 8 | |
| EA | 8 | |
| ET | 8 | |
| LP | 8 | |
| ES | 8 | |
| EH | 8 | |
| EV | 8 | |
| EP | 8 | |
| EI | 8 | |
| EL | 8 | |
| EN | 8 | |

| | non-binder | binder |
|---|---|---|
| LW | | 8 |
| LR | | 8 |
| LE | | 8 |
| ER | | 8 |
| LY | | 8 |

If L is replaced with W, R, E, or Y, binding is more likely. Even though E is very favorable for survivin binding, substituting to R is safe or even beneficial given the context. Because the context in which these mutations are introduced has a significant impact on the neural network, this analysis is clearly limited to the original 30 amino acid long sequence template and the suggested mutations cannot be generalized to any sequence. For example, if another template does not have E at all, the recommendation to substitute E to another amino acid does not even make sense.

Understanding the reaction with the target is usually insufficient when designing a peptide drug or a peptide-based diagnostic assay. It is also critical to reduce off-target protein binding and, ideally, direct the peptide against a single protein or biological function. Survivin accumulates selectively on appropriate peptides in the peptide microarray, indicating that atom composition not only directs peptides to cellular compartments, but also to specific protein environments. A network based on the protein specific binding assay can be trained when a similar peptide microarray analysis is performed on a protein other than survivin. Then you can ask very specific questions, like which mutation in my peptide increases binding to survivin while decreasing binding to, say, p53 and S100A4. Then it is possible that not all of the leucine mutations listed above will be equally plausible. The peptide can thus be targeted by an attraction/avoidance landscape defined by peptide preferences of target(s) and off-target(s) and this can be predicted virtually.

### Example Definition of a Peptide Based on Fitness Prediction

Peptides predicted by this method can be generalized by composition alone which may have to adapted to different lengths of the peptides. For example a 15-40 amino acid residue long peptide which binds the protein survivin could be described with compositions: 2.5-6.3% alanine, 0% cysteine, 30.3-35.3% aspartate, 15.0-19.2% glutamate, 0% phenylalanine, 3.7-7.1% glycine, 0.0-5.6% histidine, 0% isoleucine, 4.8%-9.1% lysine, 0% methionine, 3.3-6.4% asparagine, 0.0-5.3% proline, 3.6-6.9% glutamine, 3.2-6.3% arginine, 0% serine, 0% threonine, 0.0-4.0% tyrosine, 2.9-6.3% valine, 0% tryptophan. The composition ranges represent the composition of amino acids in peptides which have different lengths in the 15-40 range. A peptide with a fixed length can be defined by a single infinitely precise composition of individual amino acids.

Although, the present invention has been described above with reference to specific embodiments, it is not intended to be limited to the specific form set forth herein. Rather, the invention is limited only by the accompanying claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”. “an”, “first”, “second” etc do not preclude a plurality. Reference signs in the claims are provided merely as a clarifying example and shall not be construed as limiting the scope of the claims in any way.

### REFERENCES

1. Ebbing, D. D. and S. D. Gammon, General Chemistry. 2010: Houghton Mifflin.
2. Ensing, B., et al., On the origin of the extremely different solubilities of polyethers in water. Nature Communications, 2019. 10(1): p. 2893.
3. Ahlberg Gagner, V., M. Jensen, and G. Katona, Estimating the probability of coincidental similarity between atomic displacement parameters with machine learning. Machine Learning: Science and Technology, 2021. 2(3): p. 035033.
4. Gagnér, V. A., et al., Clustering of atomic displacement parameters in bovine trypsin reveals a distributed lattice of atoms with shared chemical properties. Scientific Reports, 2019. 9(1): p. 19281.
5. Lundholm, I. V., et al., Terahertz radiation induces non-thermal structural changes associated with Frohlich condensation in a protein crystal. Structural Dynamics, 2015. 2(5).
6. Fröhlich, H., Long-range coherence and energy storage in biological systems.

International Journal of Quantum Chemistry, 1968. 2(5): p. 641-649.
7. Rivadeneira, D. B., et al., Survivin promotes oxidative phosphorylation, subcellular mitochondrial repositioning, and tumor cell invasion. Sci Signal, 2015. 8(389): p. ra80.
8. Hagenbuchner, J., et al., BIRC5/Survivin enhances aerobic glycolysis and drug resistance by altered regulation of the mitochondrial fusion/fission machinery. Oncogene, 2013. 32(40): p. 4748-57.

1-20. (canceled)

21. Method for determining a fitness value of a new peptide, the fitness value corresponding to at least interaction strength with a target peptide, wherein the new peptide has not been subject to physical interaction testing with the target peptide, the method including:
generating a library of sample peptides having unique amino acid sequences,
measuring the interaction of each sample peptide with the target peptide, to determine an interaction value for each of the sample peptides,
classifying each of the sample peptides according to their atom type composition, wherein the atom type composition is based on at least one of: the type of element, number of atoms, role in a functional group, position in within the amino acid, or a combination thereof,
training a machine learning system with the sample peptides, wherein the training is based on the measured interaction and the atom type composition,
providing to the machine learning system a new peptide, not being part of the library of sample peptides, and,
predicting via the machine learning system, the fitness value of the new peptide based on the atom type composition of the new peptide.

22. The method according to claim 21, wherein the classifying of atom type composition is performed for each amino acid in a respective peptide sequence.

23. The method according to claim 21, wherein the atom type composition for each amino acid is based on each of type of element, number of atoms, role in a functional group, position within the amino acid.

24. The method according to claim 21, wherein the new peptide is classified according to its atom type composition.

25. The method according to claim 21, wherein the atom type composition comprises less than 20 categories of atom types.

26. The method according to claim 21, wherein the library of sample peptides comprises greater than 100 unique peptides, such as greater than 1000 unique peptides.

27. The method according to claim 21, wherein the atom type composition is classified according to Table 1.
TABLE 1

| aa | CA-Gly | Pro-MC | Carboxyl | Amide | His | Trp | Phe-Tyr | OH-Tyr | CH2 | CH | CH3 | OH | SH | S | NH3 | Arg | MC |
|---|---|---|---|---|---|---|---|---|---|---|---|---|---|---|---|---|---|
| A | 0 | 0 | 0 | 0 | 0 | 0 | 0 | 0 | 0 | 0 | 1 | 0 | 0 | 0 | 0 | 0 | 4 |
| C | 0 | 0 | 0 | 0 | 0 | 0 | 0 | 0 | 1 | 0 | 0 | 0 | 1 | 0 | 0 | 0 | 4 |
| D | 0 | 0 | 3 | 0 | 0 | 0 | 0 | 0 | 1 | 0 | 0 | 0 | 0 | 0 | 0 | 0 | 4 |
| E | 0 | 0 | 3 | 0 | 0 | 0 | 0 | 0 | 2 | 0 | 0 | 0 | 0 | 0 | 0 | 0 | 4 |
| F | 0 | 0 | 0 | 0 | 0 | 0 | 6 | 0 | 1 | 0 | 0 | 0 | 0 | 0 | 0 | 0 | 4 |
| G | 1 | 0 | 0 | 0 | 0 | 0 | 0 | 0 | 0 | 0 | 0 | 0 | 0 | 0 | 0 | 0 | 3 |
| H | 0 | 0 | 0 | 0 | 5 | 0 | 0 | 0 | 1 | 0 | 0 | 0 | 0 | 0 | 0 | 0 | 4 |
| I | 0 | 0 | 0 | 0 | 0 | 0 | 0 | 0 | 1 | 1 | 2 | 0 | 0 | 0 | 0 | 0 | 4 |
| K | 0 | 0 | 0 | 0 | 0 | 0 | 0 | 0 | 4 | 0 | 0 | 0 | 0 | 0 | 1 | 0 | 4 |
| L | 0 | 0 | 0 | 0 | 0 | 0 | 0 | 0 | 1 | 1 | 2 | 0 | 0 | 0 | 0 | 0 | 4 |
| M | 0 | 0 | 0 | 0 | 0 | 0 | 0 | 0 | 2 | 0 | 1 | 0 | 0 | 1 | 0 | 0 | 4 |
| N | 0 | 0 | 0 | 3 | 0 | 0 | 0 | 0 | 1 | 0 | 0 | 0 | 0 | 0 | 0 | 0 | 4 |
| P | 0 | 2 | 0 | 0 | 0 | 0 | 0 | 0 | 3 | 0 | 0 | 0 | 0 | 0 | 0 | 0 | 2 |
| Q | 0 | 0 | 0 | 3 | 0 | 0 | 0 | 0 | 2 | 0 | 0 | 0 | 0 | 0 | 0 | 0 | 4 |
| R | 0 | 0 | 0 | 0 | 0 | 0 | 0 | 0 | 3 | 0 | 0 | 0 | 0 | 0 | 0 | 4 | 4 |
| S | 0 | 0 | 0 | 0 | 0 | 0 | 0 | 0 | 1 | 0 | 0 | 1 | 0 | 0 | 0 | 0 | 4 |
| T | 0 | 0 | 0 | 0 | 0 | 0 | 0 | 0 | 0 | 1 | 1 | 1 | 0 | 0 | 0 | 0 | 4 |
| Y | 0 | 0 | 0 | 0 | 0 | 0 | 6 | 1 | 1 | 0 | 0 | 0 | 0 | 0 | 0 | 0 | 4 |
| V | 0 | 0 | 0 | 0 | 0 | 0 | 0 | 0 | 0 | 1 | 2 | 0 | 0 | 0 | 0 | 0 | 4 |
| W | 0 | 0 | 0 | 0 | 0 | 9 | 0 | 0 | 1 | 0 | 0 | 0 | 0 | 0 | 0 | 0 | 4 |

28. The method according to claim 21, wherein the machine learning system comprises a multilayer perceptron classifier.

29. The method according to claim 28, wherein the machine learning system comprises two hidden layer perceptrons.

30. The method according to claim 21, wherein the measuring of the interaction comprises classifying peptides as interacting or non-interacting based on measured fluorescence.

31. The method according to claim 21, wherein the peptide fitness corresponds to at least binding strength to the target peptide, and avoidance of an off-target peptide or peptides.

32. A method for determining atom type composition of a new peptide, the new peptide having a desired fitness corresponding to at least interaction strength with a target peptide, the method comprising
generating a library of sample peptides having unique amino acid sequences,
measuring the interaction of each sample peptide with the target peptide, to determine an interaction value for each of the sample peptides,
classifying each of the sample peptides according to their atom type composition, wherein the atom type composition is based on at least one of: the type of element, number of atoms, role in a functional group, position in within the amino acid, or a combination thereof,
training a machine learning system with the sample peptides, wherein the training is based on the measured interaction and the atom type composition,
determining via the machine learning system, the atom type composition of a new peptide having a desired fitness.

33. The method according to claim 32, wherein the fitness corresponds to the at least interaction strength with a target peptide and avoidance of an off-target peptide or peptides.

34. The method according to claim 32, wherein the classifying of atom type composition is performed for reach amino acid in a respective peptide sequence.

35. The method according to claim 32, wherein the atom type composition for each amino acid is based on each of type of element, number of atoms, role in a functional group, position within the amino acid.

36. The method according to claim 32, wherein the atom type composition comprises less than 20 categories of atom types.

37. The method according to claim 32, wherein the atom type composition is classified according to Table 1.

38. The method according to claim 32, wherein the machine learning system comprises a multilayer perceptron classifier.

39. The method according to claim 32, wherein the machine learning system comprises two hidden layer perceptrons.

40. A 15-40 amino acid residue long peptide which binds the protein survivin comprising: 2.5-6.3% alanine, 0% cysteine, 30.3-35.3% aspartate, 15.0-19.2% glutamate, 0% phenylalanine, 3.7-7.1% glycine, 0.0-5.6% histidine, 0% isoleucine, 4.8%-9.1% lysine, 0% methionine, 3.3-6.4% asparagine, 0.0-5.3% proline, 3.6-6.9% glutamine, 3.2-6.3% arginine, 0% serine, 0% threonine, 0.0-4.0% tyrosine, 2.9-6.3% valine, 0% tryptophan.
